(19) United States
(12) Patent Application Publication
(10) Pub. No.: US 2008/0131465 A1
(43) Pub. Date: Jun. 5, 2008

(54) Group a Streptococcus Crge Protein

(76) Inventor: Andrea Manetti, Siena (IT)

Correspondence Address: NOVARTIS VACCINES AND DIAGNOSTICS INC., INTELLECTUAL PROPERTY R338, P.O. BOX 8097, Emeryville, CA, 94662-8097, US

(21) Appl. No.: 11/664,019

(22) PCT Filed: Sep. 27, 2005
(86) PCT No.: PCT/IB2005/003087

§ 371 Date: Sep. 16, 2007

(30) Foreign Application Priority Data
Sep. 27, 2004 (GB) 0421465.6

(51) Int. Cl. A61K 38/16; C12N 1/20; A61K 47/06; G01N 33/53; A61P 37/00; C12Q 1/68; C07K 14/00

(52) U.S. Cl. 424/278.1; 435/253.4; 530/350; 514/12; 435/6; 435/7.1

(57) ABSTRACT

The invention provides methods of screening utilising the CrgE protein and the Streptococcus pyogenes bacterium expressing crgE. The invention also provides bacteria where the crgR and/or the crgE gene has been knocked out. The invention also provides fusion proteins comprising the polypeptide encoded by Spy1542 (crgE) and pharmaceutical compositions comprising CrgE.

All documents cited herein are incorporated by reference in their entirety.

### TECHNICAL FIELD

This invention relates to the identification and characterisation of a protein secreted by Streptococcus pyogenes that is able to inhibit cathelicidin bactericidal activity.

### BACKGROUND ART

Peptides with antimicrobial activity are found throughout nature. In mammals, antimicrobial peptides belonging to the cathelicidin family, a class of gene-encoded antibiotics, have been shown to be capable of providing an innate defensive barrier against a variety of microbial pathogens by interacting with the bacterial cellular membranes [1]. Cathelicidins are small molecules of 12-100 amino acids and share a number of properties with defensins. They are produced as a precursor consisting of an N-terminal signal peptide, a highly conserved prosequence and a structurally variable C-terminal mature peptide. Proteolytic cleavage of the inactive precursor molecule to release the mature C-terminal antimicrobial peptide is accomplished by elastase or proteinase-3 upon degranulation of activated neutrophils. About 30 different cathelicidins have been described, but so far only one species has been identified in humans (LL-37) [2] and one in mice (CRAM) [3]. The human and mouse peptides share cationic and amphipathic properties that promote favorable interactions with biological membranes. The LL-37 interaction with negatively charged membranes suggests a detergent-like effect via a ‘carpet-like’ mechanism [4]. However, other experiments support a toroidal pore mechanism of lipid bilayer disruption [5].

LL-37 and CRAMP have shown to be both highly effective against group A Streptococcus (GAS), a gram-positive extracellular bacterial pathogen which colonizes the throat or skin and is responsible for a number of suppurative infections and nonsuppurative sequelae [6]. An increased expression of LL-37 and CRAMP was described in human and murine skin after sterile incision, or in mice following infection by group A Streptococcus. Cathelicidins occur in cells directly exposed to microbial pathogens as have been detected in wounds and in mucosal epithelia of the airways [6]. LL-37 was originally isolated from human polymorphonuclear leukocytes (PMNs) [2]. In addition to its anti-microbial activity, cathelicidins have been described to have several other functions, such as induction of proteoglycan expression [7], effects on neutrophil migration and chemotactic activity [8], induction of apoptosis [9], mitogenesis and angionesesis [10]. Moreover, both LL-37 and CRAMP are present in the salivary system, in some oral epithelia, and in saliva, contributing to broad-spectrum defense of the oral cavity [11 and 12].

In a recent study Gallo and co-workers showed that the inactivation of the Streptococcus pyogenes putative GntR-like transcriptional repressor gene (crgR, for cathelicidin resistance gene regulator) elicits resistance to CRAMP and presented evidence showing an increased virulence in vivo [13]. This group, by generating transgenic mice null for Cnlp clearly demonstrated that CRAMP expression is essential in controlling GAS skin infection. In particular, after identical injections of cathelicidin-sensitive GAS in wild-type, heterozygous and homozygous-null mice, CRAMP-deficient mice were reported to develop much larger lesions. In a complementary experimental approach, CRAMP-resistant mutants were compared to wild-type and showed an increased ability to produce necrotizing cutaneous infection in mice [13].

However, the mode of action of crgR was unknown. Furthermore, the gene and the encoded protein upon which crgR acts were unknown. It is therefore an object of the invention to elucidate the protein encoded by crgR and to discover the method of action of the protein. It is a further object of the invention to provide new uses of the protein encoded by crgR and to provide a way of treating bacterial infection caused by a bacterium having resistance to CRAMP/LL-37.

### DISCLOSURE OF THE INVENTION

Identification of the crgE Gene

It has been discovered that Streptococcus pyogenes gene crgR is responsible for the regulation of a gene termed crgE. Further experiments have shown that the crgE gene maps to the open reading frame spy1542 and that the protein encoded by this gene confers resistance to cathelicidins.

Accordingly, the invention provides methods of screening utilising this protein and the Streptococcus pyogenes bacterium expressing crgE. The invention also provides bacteria where the crgR and/or the crgE gene has been knocked out. The invention also provides fusion proteins comprising the polypeptide encoded by spy1542 (crgE).

### Knockout Bacteria

The invention provides a Streptococcus bacterium in which expression of CrgR and/or CrgE has been knocked out. Preferably the bacterium is S. pyogenes. Knockout of crgR leads to derepression of CrgE expression and therefore a CRAMP/LL-37 resistant phenotype. In contrast, knockout of crgE leads to a CRAMP/LL-37 sensitive phenotype. Knockout of both crgR and crgE leads to a CRAMP/LL-37 sensitive phenotype.

The nucleotide sequence of crgR from the published genome of Streptococcus pyogenes M1 is recited as SEQ ID NO: 1 herein. The nucleotide sequence of crgE from the published genome of Streptococcus pyogenes M1 is recited as SEQ ID NO: 3 herein. The skilled person can easily identify the crgR and the crgE gene in other S. pyogenes strains based on sequence homology and the genetic environment using methods known in the art. Furthermore, the skilled person can easily identify the crgR and the crgE gene in other Streptococcus strains based on sequence homology and the genetic environment using methods known in the art. Useful markers to assist in such identification are arcB and spy1543 which flank the crgE gene in S. pyogenes at its 5′ and 3′ ends respectively.

Techniques for gene knockout are well known, and knockout mutants of Streptococcus have been reported previously (e.g. ref. 14).

The knockout is preferably achieved using allelic exchange gene replacement mutagenesis [15], but any other suitable technique may be used e.g. deletion or mutation of the promoter, deletion or mutation of the start codon, antisense inhibition, inhibitory RNA, etc. In the resulting bacterium, however, mRNA encoding the gene product of crgR and/or crgE will be absent and/or its translation will be inhibited (e.g. to less than 1% of wild-type levels).

The bacterium may contain a marker gene in place of the knocked out gene e.g. an antibiotic resistance marker.

### Polypeptides

The polypeptide encoded by crgR is recited in SEQ ID NO: 2. The polypeptide encoded by crgE is recited in SEQ ID NO: 4.

The invention provides a polypeptide comprising an amino acid sequence selected from SEQ ID NO: 2 or SEQ ID NO: 4. The invention also provides polypeptides comprising an amino acid sequence (a) having sequence identity to an amino acid sequence selected from SEQ ID NO: 2 or SEQ ID NO: 4 and/or (b) comprising a fragment of an amino acid sequence selected from SEQ ID NO: 2 or SEQ ID NO: 4. The degree of sequence identity is preferably greater than 50% (e.g. 60%, 70%, 80%, 90%, 95%, 99% or more). The fragment preferably comprises 7 or more consecutive amino acids from the reference sequence (e.g. 7, 8, 9, 10, 11, 12, 13, 14, 15, 16, 17, 18, 19, 20, 22, 24, 26, 28, 30, 32, 34, 36, 38, 40, 45, 50, 55, 60, 65, 70, 75, 80, 85, 90, 95, 100 or more). Other preferred fragments are polypeptides without their N-terminal sequences e.g. lacking 35-40 amino acids (e.g. 35, 36, 37, 38, 39 or 40) from the N-terminus. More preferably, the 38 N-terminal amino acids are deleted. The invention also provides polypeptides without their N-terminal amino acid residue.

The polypeptide recited in SEQ ID NO: 2, fragments and homologues thereof are hereafter referred to as CrgR. The polypeptide recited in SEQ ID NO: 4, fragments and homologues thereof are hereafter referred to as CrgE.

Polypeptides of the invention may be prepared by various means e.g. by chemical synthesis (at least in part), by digesting longer polypeptides using proteases, by translation from RNA, by purification from cell culture, (e.g. from recombinant expression or from S. pyogenes culture) etc.

Polypeptides are preferably prepared in a substantially pure or substantially isolated form (i.e. substantially free from other Streptococcal or host cell proteins). In general, the polypeptides are provided in a non-naturally occurring environment e.g. they are separated from their naturally occurring environment. In certain embodiments the polypeptide is present in a composition that is enriched for the polypeptide as compared to a control. As such, purified polypeptide is provided, whereby purified is meant that the polypeptide is present in a composition that is substantially free of other expressed polypeptides, whereby substantially free is meant that less than 50%, usually less than 30% and more usually less than 10% of the composition is made up of other expressed polypeptides.

The invention also provides fusion proteins comprising CrgR and/or CrgE. For example, it is often advantageous to include one or more additional amino acid sequences which may contain secretory or leader sequences, pro-sequences, sequences which aid in purification (such as glutathione-s-transferase), sequences that confer higher protein stability, for example during recombinant production or detectable labels (e.g. radioactive or fluorescent labels such as green fluorescent protein).

Such fusion proteins would have the general formula NH₂-A-B—C—COOH where -A- is an optional N-terminal sequence; —B— is a CrgR or CrgE sequence; and —C— is an optional C-terminal sequence. Each of A and C preferably independently comprises ≦20 amino acids.

Alternatively or additionally, a polypeptide of the invention may be fused with another compound at either the N-terminus or C-terminus of the polypeptide. Such compounds include those to increase the half-life of the polypeptide (for example, polyethylene glycol), compounds to ease purification of the protein (such as glutathione-s-transferase) or detectable labels (e.g. radioactive or fluorescent labels such as green fluorescent protein).

### Mutant Enzyme

The invention also provides mutant CrgE enzyme, where one or more amino acids at the active site have been altered such that the enzyme can no longer bind its substrate. Such mutations may be carried out by any one of a number of methods known in the art, for example by random mutagenesis. Random mutagenesis may be induced chemically or exposure to radiation. CrgE molecules mutagenised in this way may then be screened to detect those mutants which are no longer functional.

Use of the crgE Gene Product

As cathelicidins have been shown to play a role in tissue repair [16], it is possible that they also play a role in graft rejection. Therefore the downregulation of cathelicidins may reduce the level of graft rejection. Downregulation of secreted cathelicidin activity by CrgE may be one way of reducing graft rejection. The invention therefore provides the use of CrgE in therapy. The invention further provides a method of preventing and/or treating graft rejection comprising administering CrgE to a subject at risk of graft rejection. The invention also provides the use of CrgE in the manufacture of a medicament for the prevention and/or treatment of graft rejection. Graft rejection may occur in mammals, said mammal is preferably a human. The subject at risk of graft rejection may be an organ recipient and may further be a smoker.

Similarly, the cathelicidin LL-37 has been shown to kill vaccinia virus [17] and to have inhibitory activity against other enveloped viruses [16]. This has implications for vaccination strategies as the vaccine may be destroyed by the innate immune response before the adaptive arm of the immune system has a chance to respond. It may be possible to use CrgE to reduce the LL-37 activity against such viral vaccines. The invention therefore provides the use of CrgE as an adjuvant. The invention also provides a method of enhancing vaccination comprising administering both vaccine and CrgE to a subject. Said subject is a mammal, preferably a human and is most preferably a non- or hypo-responder to the vaccine. The invention further comprises vaccine compositions comprising (a) a vaccine and (b) a CrgE polypeptide.

### Screening Methods

The invention provides a process for determining whether a test compound down-regulates expression of a target polypeptide, comprising the steps of: (a) contacting the test compound with a S. pyogenes bacterium to form a mixture; (b) incubating the mixture to allow the compound and the bacterium to interact; and (c) determining whether expression of the target polypeptide is down-regulated. The compound may act by inhibiting transcription or translation.

The invention also provides a process for determining whether a test compound binds to a target polypeptide, comprising the steps of: (a) contacting the test compound with the target polypeptide to form a mixture; (b) incubating the mixture to allow the compound and the target polypeptide to interact; and (c) determining whether the compound and polypeptide interact.

Where a target polypeptide is an enzyme (e.g. CrgE), the invention also provides a process for determining whether a test compound inhibits the enzymatic activity of a target polypeptide, comprising the steps of: (a) contacting the test compound with the target polypeptide and a substrate for the enzymatic reaction catalysed by the target polypeptide; (b) incubating the mixture to allow the compound, target polypeptide and substrate to interact; and (c) determining whether modification of the substrate by the enzymatic activity is inhibited by the test compound.

The invention also provides a method for detecting a bacterium expressing the polypeptides CrgR or CrgE. Such a method may comprise the steps of: (a) contacting a S. pyogenes bacterium with an antibody specific for CrgR or CrgE; (b) incubating the mixture to allow the antibody and bacterium to interact; and (c) detecting binding of the antibody to the bacterium. Alternatively, such a method may comprise the steps of: (a) contacting a S. pyogenes bacterium with a first antibody specific for CrgR or CrgE; (b) incubating the mixture to allow the first antibody and bacterium to interact; (c) contacting the mixture with a second labelled antibody specific for the first antibody; and (d) detecting binding of the second labelled antibody to the first antibody.

The target polypeptide in the methods is preferably the S. pyogenes polypeptide CrgR or CrgE. As an alternative, the polypeptide may be the homologue of a polypeptide encoded by crgR or crgE from another Streptococcus (such as S. pneumoniae or S. agalactiae) or from another Gram-positive bacterium.

The test compound may be of extracellular or intracellular and may have a biologic or chemical origin. Typical test compounds include peptide, peptoids, lipids, nucleotides, nucleosides, small organic molecules, antibiotics, polyamines, polymers, or derivatives thereof. Small organic molecules have a molecular weight of between 50 and 2500 Da, and most preferably between about 300 and about 800 Da.

The test compound may be in a purified form, or may be part of a mixture of substances, such as extracts containing natural products, or the products of mixed combinatorial syntheses. Test compounds may be derived from large libraries of synthetic or natural compounds. For instance, synthetic compound libraries are commercially available, as are libraries of natural compounds in the form of bacterial, fungal, plant and animal extracts. If a mixture is found to have a useful activity then that activity can then be traced to specific component(s) either by knowing the components and testing them individually, or by purification or deconvolution. Additionally, test compounds may be synthetically produced using combinatorial chemistry either, as individual compounds or as mixtures.

The screening method of the invention is preferably arranged in a high-throughput format. Conveniently, the method is performed in a microtitre plate.

If a test compound binds to CrgE and this binding inhibits the resistance to cathelicidin activity of the S. pyogenes, bacterium, then the test compound can be used to assist in antimicrobial therapy. Alternatively, if a test compound binds to CrgR and this binding upregulates the repression of CrgE, then the test compound can be used to assist in antimicrobial therapy.

Methods for detecting down-regulation of transcription are well known in the art, and the method of detection is not critical to the invention. Methods for detecting mRNA include, but are not limited to amplification assays such as quantitative RT-PCR, and/or hybridisation assays such as Northern analysis, dot blots, slot blots, in situ hybridisation, DNA assays, microarray, etc.

Methods for detecting down-regulation of translation are also well known in the art and, again, the method of detection is not critical to the invention. Methods of polypeptide detection include, but are not limited to, immunodetection methods such as Western blots, ELISA assays, polyacrylamide gel electrophoresis, mass spectroscopy, and enzymatic assays.

Methods for detecting a binding interaction are well known in the art and may involve techniques such as NMR, filter-binding assays, gel-retardation or gel-shift assays, displacement assays, western blots, radiolabeled competition assays, co-fractionation by chromatography, co-precipitation, cross linking, surface plasmon resonance, reverse two-hybrid, etc. A compound which is found to bind to a polypeptide can be tested for its use in antimicrobial therapy by contacting the compound with S. pyogenes (or another bacterium), contacting with a cathelicidin and then monitoring for inhibition of growth or inhibition of virulence determinants.

Direct methods for detecting a binding interaction may involve a labelled test compound and/or polypeptide. The label may be a fluorophore, radioisotope, or other detectable label. Association of the label with the polypeptide indicates a binding interaction. Other direct methods for assessing interaction between the test compound and a target polypeptide may include using NMR to determine whether a polypeptide:compound complex is present.

Another method of assessing interaction between a polypeptide and a test compound may involve immobilising the polypeptide on a solid surface and assaying for the presence of free test compound. If there is no interaction between the test compound and the polypeptide then free test compound will be detected. The test compound may be labelled to facilitate detection. This type of assay may also be carried with the test compound being immobilised on the solid surface. Interaction between the immobilised polypeptide and the free test compound may also be monitored by a process such as surface plasmon resonance.

Methods for assessing inhibition of enzymatic activity are well known [e.g. ref. 18]. Enzyme substrates are widely available from commercial manufacturers, including those adapted for in vitro assays e.g. coloured substrates or products to give visible indications of enzymatic activity, etc.

In the methods of the invention, a reference standard is typically needed in order to detect whether a target polypeptide and a test compound interact, or to detect whether expression of a given target polypeptide has been inhibited, or to detect whether enzymatic activity is inhibited.

One standard is a control experiment run in parallel to a process of the invention in the absence of the test compound. The results achieved in the control experiment and the process of the invention can then be compared in order to assess the effect of the test compound. As an alternative to determining the standard in parallel, it may have been determined before performing the process of the invention, or after the process has been performed. The standard may be an absolute standard derived from previous work.

Some embodiments of the invention comprise using competitive screening assays in which neutralising antibodies capable of binding a target polypeptide specifically compete with a test compound for binding to the polypeptide. In this manner, the antibodies can be used to detect the presence of any peptide which shares one or more antigenic determinants with the S. pyogenes polypeptide. Radiolabelled competitive binding studies are described in ref. 19.

In other embodiments, the S. pyogenes polypeptides are employed as research tools for identification, characterisation and purification of interacting, regulatory proteins. Appropriate labels are incorporated into the polypeptides of the invention by various methods known in the art and the polypeptides are used to capture interacting molecules. For example, molecules are incubated with the labelled polypeptides, washed to remove unbound polypeptides, and the polypeptide complex is quantified. Data obtained using different concentrations of polypeptide are used to calculate values for the number, affinity, and association of polypeptide with the complex.

### Compounds Identified by Screening Processes

Test compounds which down-regulate expression of and/or which bind to a target polypeptide and/or which inhibit an enzymatic activity may be useful as antibiotics, antibiotic candidates, or lead compounds for antibiotic development. Such a test compound may act by enhancing the activity of CrgR, thus repressing CrgE expression or by binding CrgE and repressing its activity.

Once a test compound has been identified as a compound that binds to a target polypeptide, or which inhibits its expression in a bacterium, it may be desirable to perform further experiments to confirm the in vivo function of the compound in inhibiting bacterial growth. Any of the above processes may therefore comprise the further steps of contacting the test compound with a bacterium and assessing its effect on bacterial growth and/or survival. Methods for determining bacterial growth and survival are routinely available.

The invention provides a compound obtained or obtainable by any of the processes described above. Preferably, the compounds are organic compounds.

Once a compound has been identified using a process of the invention, it may be necessary to conduct further work on its pharmaceutical properties. For example, it may be necessary to alter the compound to improve its pharmacokinetic properties or bioavailability. The invention extends to any compounds identified by the methods of the invention which have been altered to improve their pharmacokinetic properties and/or bioavailability, and to compositions comprising those compounds.

The invention further provides compounds obtained or obtainable using the processes of the invention, and compositions comprising those compounds, for use as a medicament e.g. as an antibiotic. The invention also provides the use of compounds obtained or obtainable using the processes of the invention in the manufacture of an antibiotic, particularly an antibiotic for treating S. pyogenes infection.

The invention also provides a method for producing an antibiotic composition, comprising the steps of: (a) identifying a compound as described above; (b) manufacturing the compound; (c) formulating the compound for administration to a patient; and (d) packaging the formulated compound to produce the antibiotic composition. Details of pharmaceutical formulation can be found in ref. 20.

### Compositions

The invention also provides pharmaceutical compositions comprising CrgE or a homologue from another Streptococcus.

Compositions of the invention are immunogenic, and are more preferably vaccine compositions.

Vaccines according to the invention may either be prophylactic (i.e. to prevent infection) or therapeutic (i.e. to treat infection), but will typically be prophylactic.

The pH of the composition is preferably between 6 and 8, preferably about 7. Stable pH may be maintained by the use of a buffer. The composition may be sterile and/or pyrogen-free. Compositions of the invention may be isotonic with respect to humans.

Compositions may be presented in vials, or they may be presented in ready-filled syringes. The syringes may be supplied with or without needles. A syringe will include a single dose of the composition, whereas a vial may include a single dose or multiple doses. Injectable compositions will usually be liquid solutions or suspensions. Alternatively, they may be presented in solid form (e.g. freeze-dried) for solution or suspension in liquid vehicles prior to injection.

Compositions of the invention may be packaged in unit dose form or in multiple dose form. For multiple dose forms, vials are preferred to pre-filled syringes. Effective dosage volumes can be routinely established, but a typical human dose of the composition for injection has a volume of 0.5 ml.

Where a composition of the invention is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

The invention also provides a composition of the invention for use as a medicament. The medicament is preferably able to raise an immune response in a mammal (i.e. it is an immunogenic composition) and is more preferably a vaccine.

The invention also provides the use of CrgE (and other optional antigens) in the manufacture of a medicament for raising an immune response in a mammal. The medicament is preferably a vaccine.

The invention also provides a method for raising an immune response in a mammal comprising the step of administering an effective amount of a composition of the invention. The immune response is preferably protective and preferably involves antibodies. The method may raise a booster response.

The mammal is preferably a human. Where the vaccine is for prophylactic use, the human is preferably a child (e.g. a toddler or infant); where the vaccine is for therapeutic use, the human is preferably an adult. A vaccine intended for children may also be administered to adults e.g. to assess safety, dosage, immunogenicity, etc.

These uses and methods are preferably for the prevention and/or treatment of a disease caused by a Streptococcus (e.g. pneumonia, septicaemia, bacteremia etc.).

One way of checking efficacy of therapeutic treatment involves monitoring Streptococcal infection after administration of the composition of the invention. One way of checking efficacy of prophylactic treatment involves monitoring immune responses against the antigen after administration of the composition. Immunogenicity of compositions of the invention can be determined by administering them to test subjects (e.g. children 12-16 months age, or animal models [21]) and then determining standard parameters including ELISA titres (GMT) of antibody.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is 0.5 ml.

The invention may be used to elicit systemic and/or mucosal immunity.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

Streptococcal infections affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition). The composition may be prepared for topical administration e.g. as an ointment, cream or powder. The composition may be prepared for oral administration e.g. as a tablet or capsule, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as spray, drops, gel or powder [e.g. refs 22 & 23].

Immunogenic compositions used as vaccines comprise an immunologically effective amount of antigen(s), as well as any other components, as needed. By ‘immunologically effective amount’ it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

Antigens which may be included in compositions of the invention include, but are not limited to:
an outer-membrane vesicle (OMV) preparation from N. meningitidis serogroup B, such as those disclosed in refs. 24-27 etc.
a saccharide antigen from N meningitidis serogroup A, C, W135 and/or Y, such as the oligosaccharide disclosed in ref. 28 from serogroup C [see also ref. 29] or the oligosaccharides of ref. 30.
a protein antigen from N. meningitidis serogroup B, such as those disclosed in refs. 31-39, etc.
antigens from Helicobacter pylori such as CagA [40 to 43], VacA [44, 45], NAP [46, 47, 48], HopX [e.g. 49], HopY [e.g. 49] and/or urease.
a saccharide antigen from Streptococcus pneumoniae [e.g. 50, 51, 52].
an antigen from hepatitis A virus, such as inactivated virus [e.g. 53, 54].
an antigen from hepatitis B virus, such as the surface and/or core antigens [e.g. 54, 55].
an antigen from hepatitis C virus [e.g. 56].
an antigen from HIV [57]
a diphtheria antigen, such as a diphtheria toxoid [e.g. chapter 3 of ref. 58].
a tetanus antigen, such as a tetanus toxoid [e.g. chapter 4 of ref. 58].
an antigen from Bordetella pertussis, such as pertussis holotoxin (PT) and filamentous haemagglutinin (FHA) from B. pertussis, optionally also in combination with pertactin and/or agglutinogens 2 and 3 [e.g. refs. 59 & 60].
a saccharide antigen from Haemophilus influenzae B [e.g. 29].
polio antigen(s) [e.g. 61, 62] such as IPV.
an antigen from N. gonorrhoeae [e.g. 63, 64, 65, 66].
an antigen from Chlamydia pneumoniae [e.g. refs. 67 to 73].
an antigen from Chlamydia trachomatis [e.g. 74].
an antigen from Porphyromonas gingivalis [e.g. 75].
rabies antigen(s) [e.g. 76] such as lyophilised inactivated virus [e.g. 77, RabAvert™].
measles, mumps and/or rubella antigens [e.g. chapters 9, 10 & 11 of ref. 58].
influenza antigen(s) [e.g. chapter 19 of ref. 58], such as the haemagglutinin and/or neuraminidase surface proteins. The flu antigen may be selected from a pandemic strain.
antigen(s) from a paramyxovirus such as respiratory syncytial virus (RSV [78, 79]) and/or parainfluenza virus (PIV3 [80]).
an antigen from Moraxella catarrhalis [e.g. 81].
an antigen from Streptococcus pyogenes (group A streptococcus) [e.g. 82, 83, 84].
an antigen from Staphylococcus aureus [e.g. 85].
an antigen from Bacillus anthracis [e.g. 86, 87, 88].
an antigen from a virus in the flaviviridae family (genus flavivirus), such as from yellow fever virus, Japanese encephalitis virus, four serotypes of Dengue viruses, tick-borne encephalitis virus, West Nile virus.
a pestivirus antigen, such as from classical porcine fever virus, bovine viral diarrhoea virus, and/or border disease virus.
a parvovirus antigen e.g. from parvovirus B19.
a coronavirus antigen, such as a SARS coronavirus antigen [89]
a Norwalk virus antigen [90]
a prion protein (e.g. the CJD prion protein)
an amyloid protein, such as a beta peptide [91]
a cancer antigen, such as those listed in Table 1 of ref. 92 or in tables 3 & 4 of ref. 93
an allergen that triggers an allergic or asthmatic response

### Further Non-Antigen Components of the Composition

The composition of the invention will typically, in addition to the components mentioned above, comprise one or more ‘pharmaceutically acceptable carriers’, which include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, lactose, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in reference 20.

Compositions of the invention may include an antimicrobial, particularly when packaged in; multiple dose format.

Compositions of the invention may comprise detergent e.g. a Tween (polysorbate), such as Tween 80. Detergents are generally present at low levels e.g. <0.01%.

Compositions of the invention may include sodium salts (e.g. sodium chloride) to give tonicity. A concentration of 10±2 mg/ml NaCl is typical.

Compositions of the invention will generally include a buffer. A phosphate buffer is typical.

### DEFINITIONS

The term “comprising” encompasses “including” as well as “consisting” e.g. a composition “comprising” X may consist exclusively of X or may include something additional e.g. X+Y.

The term “about” in relation to a numerical value x means, for example, x+10%.

The word “substantially” does not exclude “completely” e.g. a composition which is “substantially free” from Y may be completely free from Y. Where necessary, the word “substantially” may be omitted from the definition of the invention.

The term “heterologous” refers to two biological components that are not found together in nature. The components may be host cells, genes, or regulatory regions, such as promoters.

Although the heterologous components are not found together in nature, they can function together, as when a promoter heterologous to a gene is operably linked to the gene. Another example is where a Streptococcus sequence is heterologous to a mouse host cell. A further example would be two epitopes from the same or different proteins which have been assembled in a single protein in an arrangement not found in nature.

By “antibiotic” we refer to a compound or composition that can destroy or inhibit the growth of bacteria or can downregulate virulence determinants.

By “antimicrobial” we mean capable to destroy or inhibit the growth of bacteria or down-regulate virulence determinants.

An “adjuvant” is a pharmacological agent added to a drug to increase or aid its effect or an immunological agent that increases the antigenic response.

### BRIEF DESCRIPTION OF DRAWINGS

FIG. 1 shows bacterial killing assays for both (A) CRAMP and (B) LL-37.

FIG. 2 shows a putative palindromic crgR operator sequence between arcB and spy1543.

FIG. 3 shows transcriptional profiling of the arc operon.

FIG. 4 shows a western blot demonstrating the inactivation of spy1542 in the ΔcrgR/Δspy1542 mutant.

FIG. 5 shows a) the response of the wild type and mutant strains to serial dilutions of LL-37 and b) the response of the wild type and mutant strains to 0.2 μM LL-37.

FIG. 6 shows the response of the wild type and mutant strains to serial dilutions of CRAMP.

FIG. 7 shows the confirmation of the expression of Spy1542 following cloning and expression as a native protein.

FIG. 8 shows a) the response of the wild type and mutant strains to challenge with differing ratios of LL-37 and Spy1542 and b) the response of the wild type and mutant strains to challenge with differing ratios of CRAMP and Spy1542.

FIG. 9 shows legions in ΔcrgR and ΔcrgR/Δspy1542-1543 mice compared to those in wt mice.

FIG. 10 shows the results of the bacterial killing assay carried out on. Streptococcus pneumoniae.

FIG. 11 shows the presence of Spy 1542 in the supernatant of the WT bacterial strain, but not in the mutant strains. The lanes are: A) WT, B) ΔCrgR/ΔSpy1542-1543, C) ΔCrgR1, D) ΔCrgR2

FIG. 12 gives the PSORT output (http://psort.nibb.ac.jp/) when queried with the CrgE polypeptide sequence.

FIG. 13 shows the mass spectrometry results of LL-37 with (A) and without (B) treatment with Spy1542.

FIG. 14 shows the FACS analysis of the WT and ΔSpy1542 strains treated with 0 μM or 0.8 μM LL-37 for 5 or 10 minutes. Conditions in each graph is as follows: A) WT, 5 min @ 37° C., B) ΔSpy1542 5 min @ 37° C., C) WT, 10 min @ 37° C. and D) ΔSpy1542 10 min @ 37° C.

FIG. 15 shows the protective effect of pre-treating bacteria with Spy1542 before washing and challenge with LL-37.

FIG. 16 shows the arrangement of genes in arc operon in the knockout mutant.

### MODES FOR CARRYING OUT THE INVENTION

### Example 1

### Identification of Putative crgR Regulated Genes by Transcriptome Comparison of the GAS Strain Sf370 with its ΔcrgR Mutant

In order to identify the genes responsible for resistance phenotype a transcriptome comparison of the GAS strain SF370 with its ΔcrgR mutant was performed.

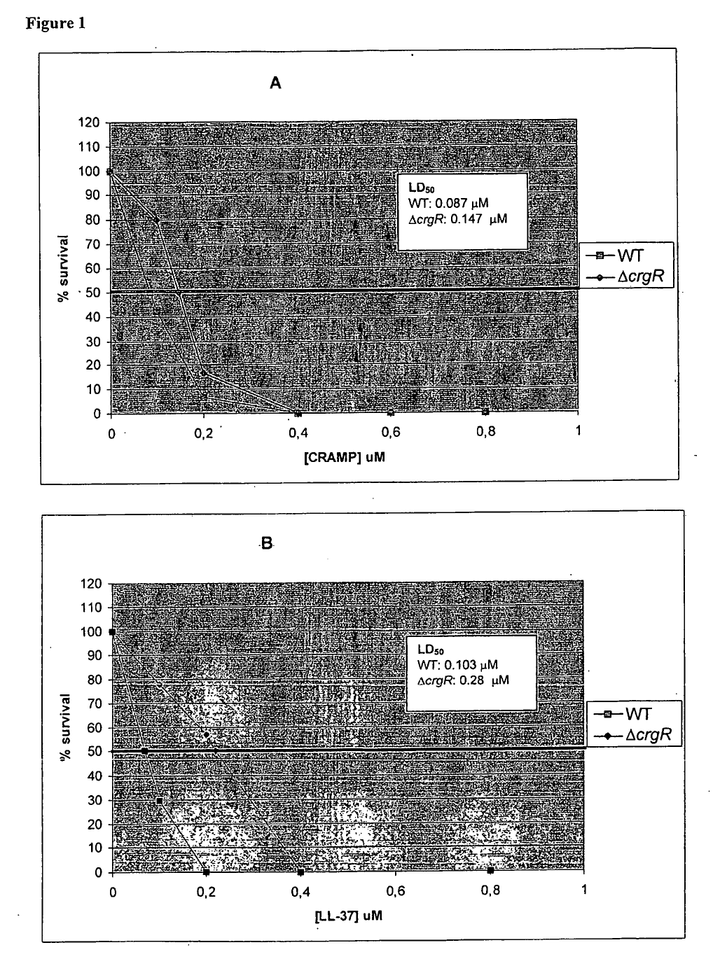

The mutant was obtained using allelic exchange gene replacement mutagenesis [15]. Positive clones were confirmed by PCR and used for further experiments. Quantitative real time PCR confirmed the absence of crgR mRNA in the mutant. ΔcrgR mutant cathelicidin resistance was tested in a bacterial killing assay. Bacteria were grown in THB to OD600 0.2 and diluted in 10 uM Tris-HCl, pH7.5, containing 5 mM glucose. 1 ml of bacteria (˜106 cfu) were incubated together with different concentrations of cathelicidins for 1 hour at 37° C. with no agitation. The bactericidal activity was assessed by plating serial dilutions of the incubation mixtures on THY-blood agar. Under the experimental conditions used, the concentration of LL-37 necessary to kill 50% of bacterial cells (LD₅₀) was 0.103 μM for WT and nearly three fold higher (0.28 μM) for the ΔcrgR mutant (FIG. 1). The LD₅₀ of CRAMP resulted to be 0.087 μM for WT, whereas it was 0.147 μM for the ΔcrgR mutant (FIG. 1). These data confirm crgR involvement in both CRAMP and LL-37 resistance.

Total bacterial RNA extracted from exponentially growing mutant cells was used for complementary DNA synthesis, labeled with Cy-5 (or Cy-3) and hybridized to the microarray in the presence of Cy-3 (or Cy-5)-labeled common reference (exponentially growing wild-type SF370 strain). Each sample was hybridized in duplicate and the relative fluorescent intensities determined. Data were quantified, normalized and corrected to yield relative transcript abundance of each gene as an intensity ratio with respect to that of the reference signal. Although mRNA changes as low as 1.5-fold may have biological relevance (Hughes et al., 2000), the more stringent threshold of 2-fold was used here.

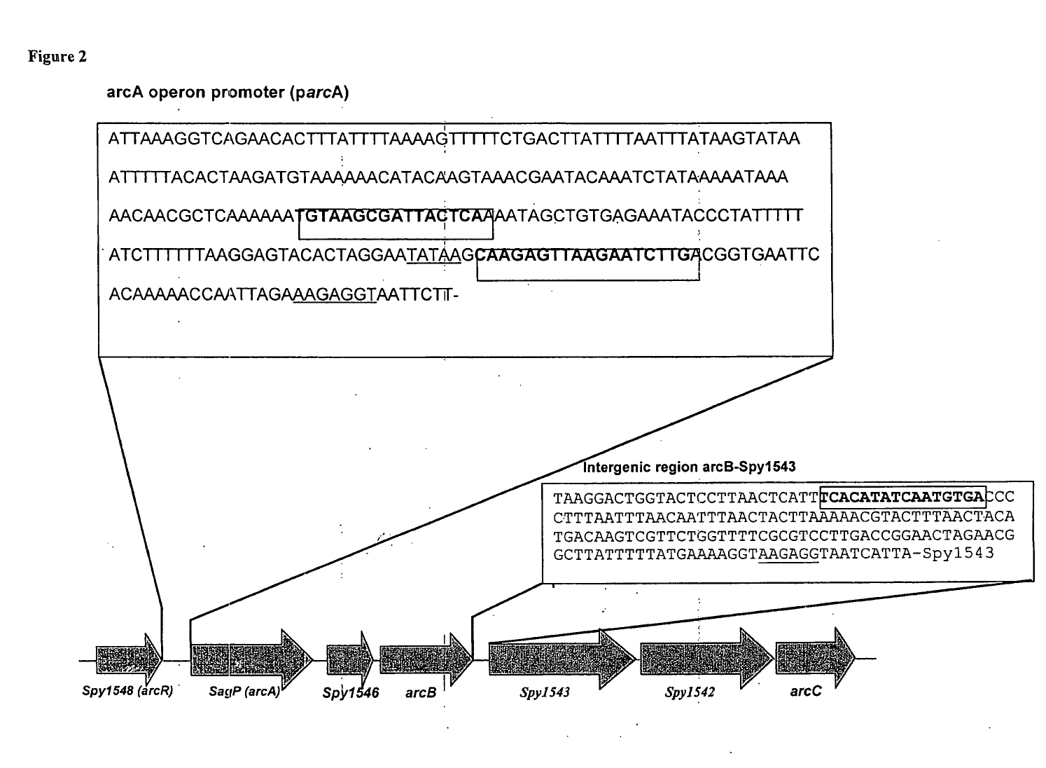

As reported in Table 1, gene members of 11 transcription units showed an expression fold-change higher than 2 in the ΔcrgR mutant. 8 transcriptional units out of the 11 were up-regulated and 3 down-regulated (including crgR itself). A putative palindromic operator sequence spanning position −59 to −42 in the promoter region of arc operon (arcAp) was identified. This putative consensus sequence closely resembles those related to members of GntR family (Rigali et al., 2002). Another putative palindromic crgR operator sequence was found in the intergenic region between arcB and spy1543, spanning positions −144 to −129 (FIG. 2). Further sequence analysis identified a consensus sequence spanning positions −135 to −120 (FIG. 2) with high homology to a cre element (catabolite-responsive element). This result is consistent with the hypothetical function of crgR gene as a GntR-like transcriptional repressor. Members of the GntR family have in fact been shown to be involved in the regulation of oxidized substrates related to amino acids metabolism or at the crossroads of various metabolic pathways such as aspartate (AnsR), pyruvate (PdhR), glycolate (GlcC), galactonate (DgoR), lactate (LldR), malonate (MatR), or gluconate (GntR) (Rigali et al., 2002).

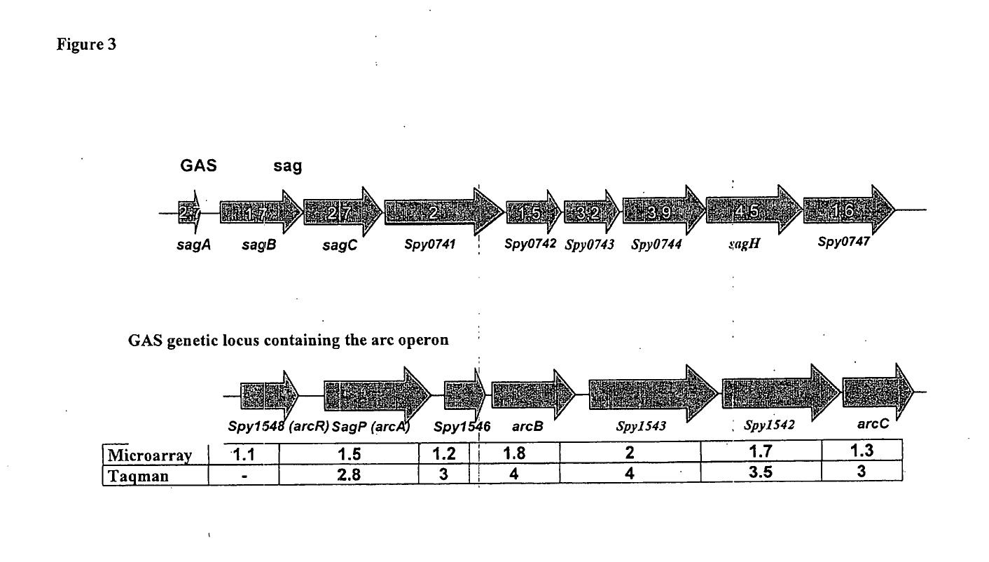

Among the regulated genes, spy1542 of the arc operon (FIG. 3) was first selected for further investigation.

Although in the case of the arc operon DNA microarray transcriptional profiling identified just one gene which was significantly up-regulated in the ΔcrgR mutant, the actual up-regulation of the entire operon was confirmed by quantitative real time PCR (the expression fold-change values are shown in (FIG. 3)). Based on these observations it was decided to further investigate the possible involvement of arc operon spy1542 in cathelicidin resistance.

### Example 2

### Spy1542 is Effectively Involved in Cathelicidin Resistance

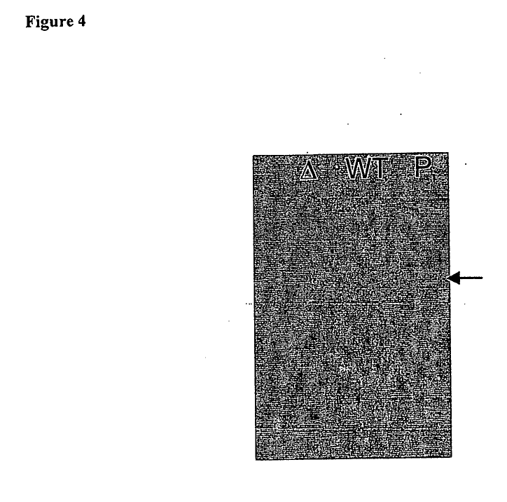

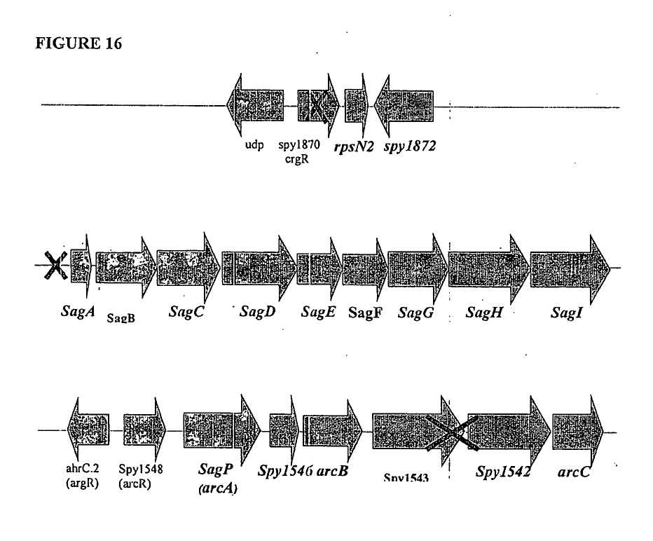

ΔcrgR/Δspy1542 mutant (FIG. 16) was obtained by mutating ΔcrgR electro-competent cells. Δspy1542 mutant was produced by deleting 539 bp of the final portion of spy1543 coding for the last 180 amino acids, an intragenic region of 16 bp and 69 bp located at the 5′ end of spy1542 which codes for the first 23 amino acids (see Table 2). Inactivation of the spy1542 gene in the mutant was verified by western blot on total protein extract of both WT and ΔcrgR/Δspy1542-43 strains, using sera from mice immunized with the purified Spy1542 protein. A signal of the expected size (49 KDa) was detectable in WT but not in ΔcrgR/Δspy1542-43 lane (FIG. 4).

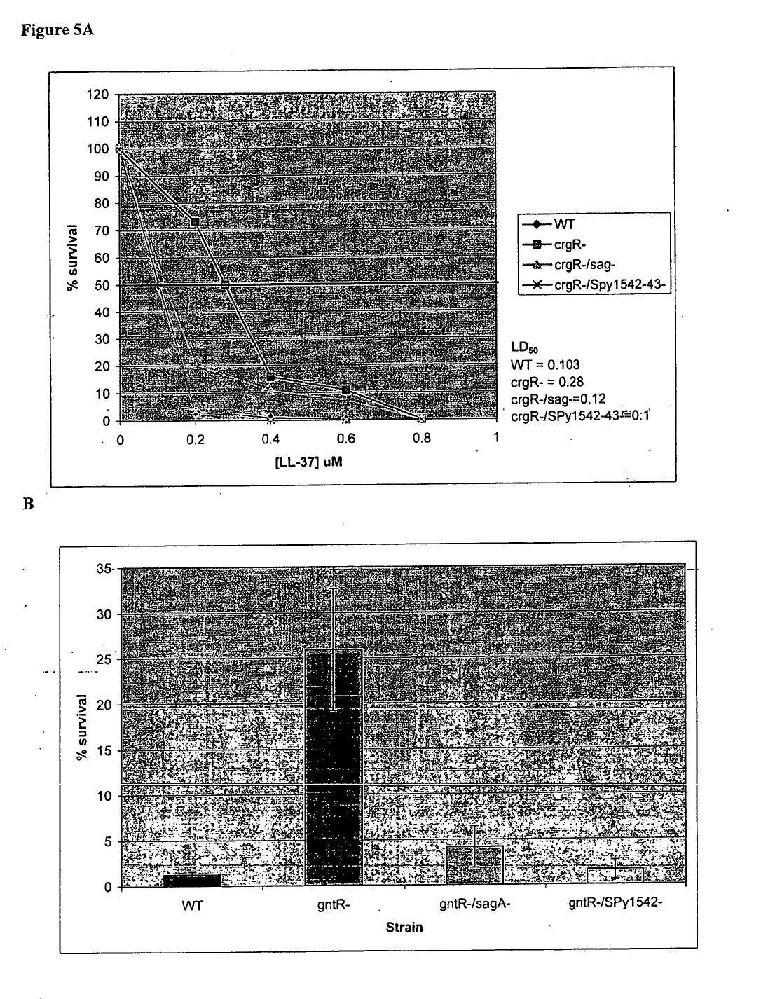

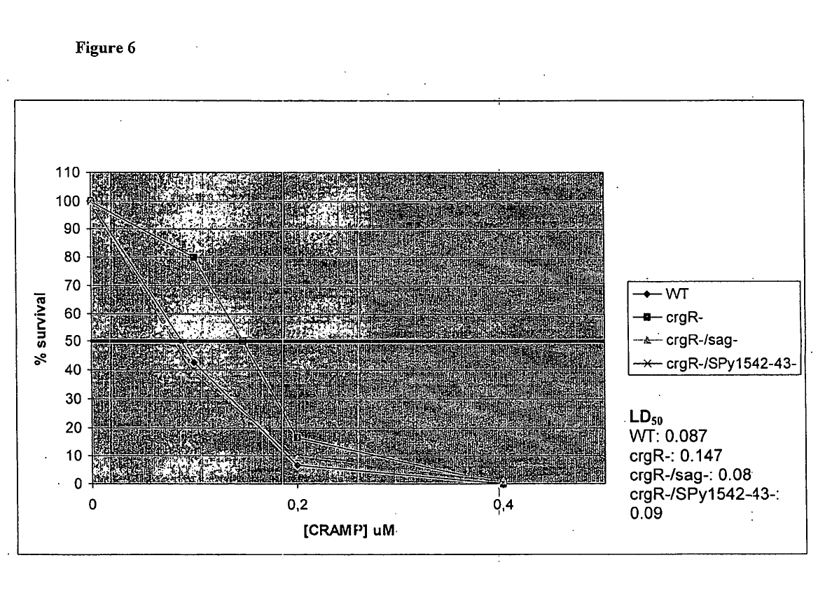

In order to investigate the behavior of the new mutant in the presence of LL-37, a new bacterial killing experiment was performed. Exponentially growing WT, ΔcrgR and ΔcrgR/Δspy1542-43 mutant cells were challenged with different amounts of LL-37 and serial dilutions were plated for colony count. As reported in FIG. 5a, LD₅₀ value of ΔcrgR/Δspy1542-43 mutant reverted to WT. Survival rates in the presence of 0.2 μM LL-37 (FIG. 5b) showed that the mutant bearing inactivated spy1542 and spy1543 genes revert to sensitivity to LL-37. Similar conclusions were drawn by repeating bacterial killing experiments with the murine cathelicidin CRAMP (FIG. 6).

### Example 3

### Spy1542 Protein Product Neutralizes LL-37 Antibacterial Action In Vitro

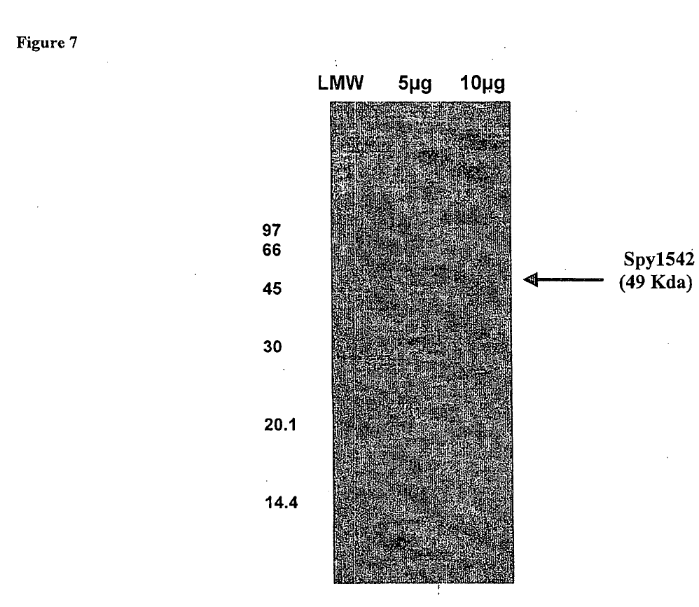

The involvement of Spy1542 in the resistance mechanism to antimicrobial peptides was investigated further by cloning and expressing Spy1542 as a native protein. The expressed protein was purified by using a Q sepharose column followed by Cu chelating and finally, gel filtration (FIG. 7). The purified protein was successively used to study its effect when added to the medium in bacterial-killing assays.

Wild-type; ΔcrgR and ΔcrgR/Δspy1542-43 strains were challenged with 0.4 μM LL-37 or CRAMP, and added with different concentrations of the purified protein.

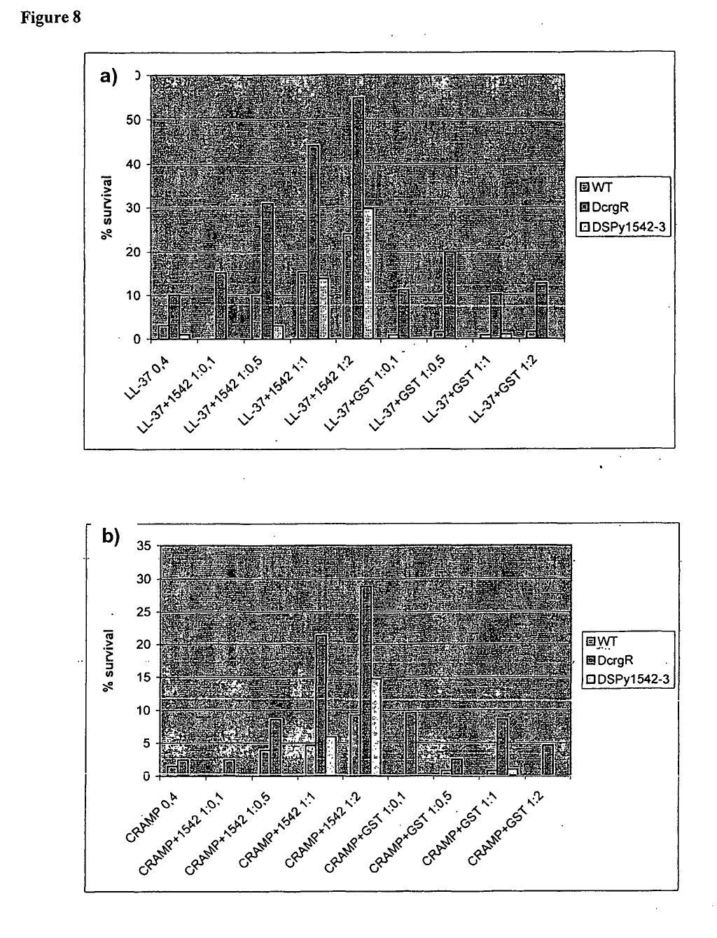

As shown in FIG. 8a, Spy1542 protein inhibits LL-37 action in a dose dependent manner. An increase in the number of colonies was observed for both WT and its ΔcrgR/Δspy1542-43 mutant, when an increasing amount of Spy1542 was added. In addition, Spy1542 protein was also able to enhance in a dose dependent manner the survival rate of the ΔcrgR mutant (FIG. 8a). The experiment was repeated with CRAMP and, again, Spy1542 inhibited cathelicidin bactericidal activity in a dose-dependent fashion (FIG. 8b).

### Example 4

### ΔcrgR Mutant but not ΔcrgR/ΔSpy1542-43 Mutant, Inhibits Cathelicidin Action In Vivo

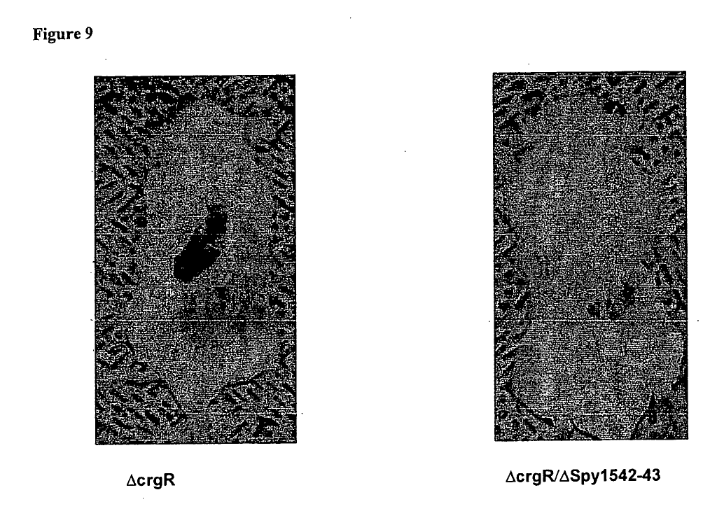

The crgR/Δspy1542-43 mutant was compared to the ΔcrgR mutant and wild-type GAS for its ability to produce necrotizing cutaneous infection in a mouse model. Three groups of 8 CD1 female mice were injected subcutaneously with 10⁸ CFU of wild-type, ΔcrgR or ΔcrgR/Δspy1542-43 strains and observed daily for skin lesions. As previously shown by Gallo and co-workers, the ΔcrgR mutant confirmed its ability to generate larger cutaneous lesions when compared to WT (FIG. 9; Table 3). The experiment also demonstrates that mice infected with ΔcrgR/Δspy1542-43 mutant strain had lesions of smaller size than those infected with the wild-type GAS strain. The average ΔcrgR lesion size was more than 13-fold larger than the ΔcrgR/Δspy1542-43 generated lesions (FIG. 9).

### Example 5

### Action on Heterologous Proteins

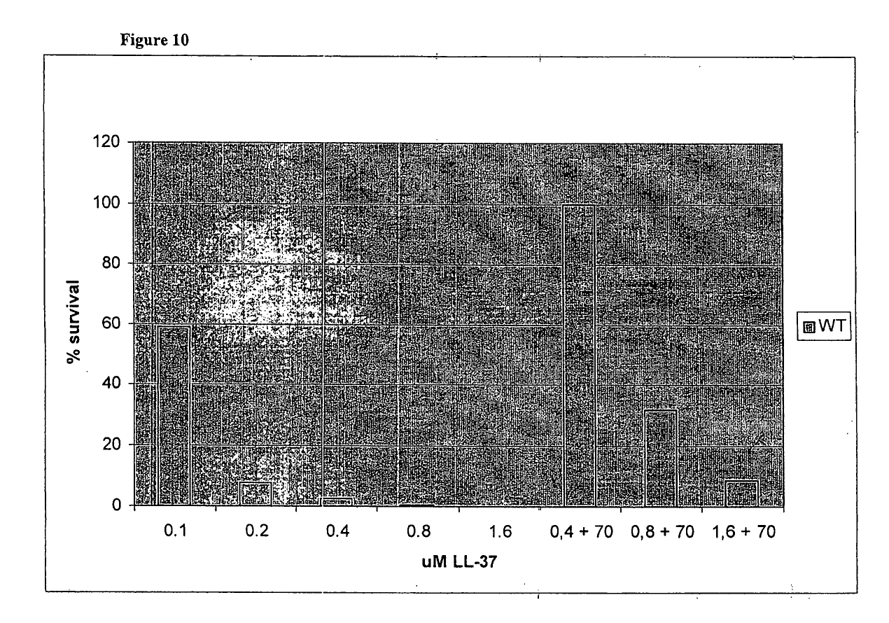

In order to see if a similar mechanism of action was found in other bacteria encoding heterologous proteins, the antimicrobial assay was carried out as described above, except using Streptococcus pneumoniae. As before, the bacteria were subjected to increasing concentrations of LL-37 which were shown to kill the bacteria (see FIG. 10). The experiment was repeated by adding 70 μg of native Spy1542 in addition to 0.4 μM, 0.8 μM or 1.6 μM of LL-37. In this second experiment, 100% survival at 0.4 μM was noted. Therefore Spy1542 appears to be able to inhibit cathelicidin action also in heterologous systems

### Example 6

### Mode of Action of Spy1542

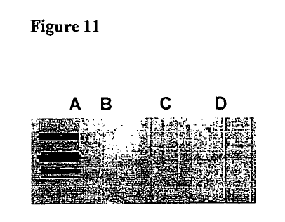

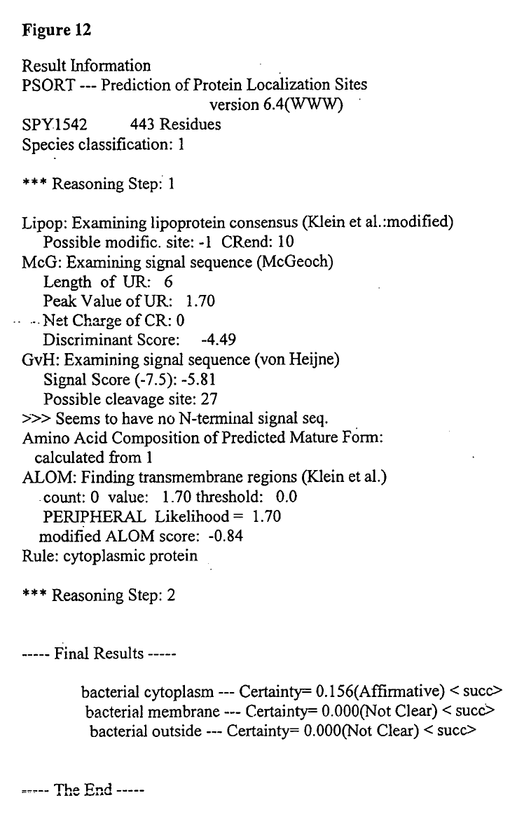

Spy1542 has been shown to be a secreted protein (see FIG. 11). As the results show, Spy1542 appears to be present in the supernatant of the WT strain, but not in that of the mutant strain. This is surprising as using PSORT (http://psort.nibb.ac.jp/), it is predicted that the protein is actually found in the bacterial cytoplasm (see FIG. 12).

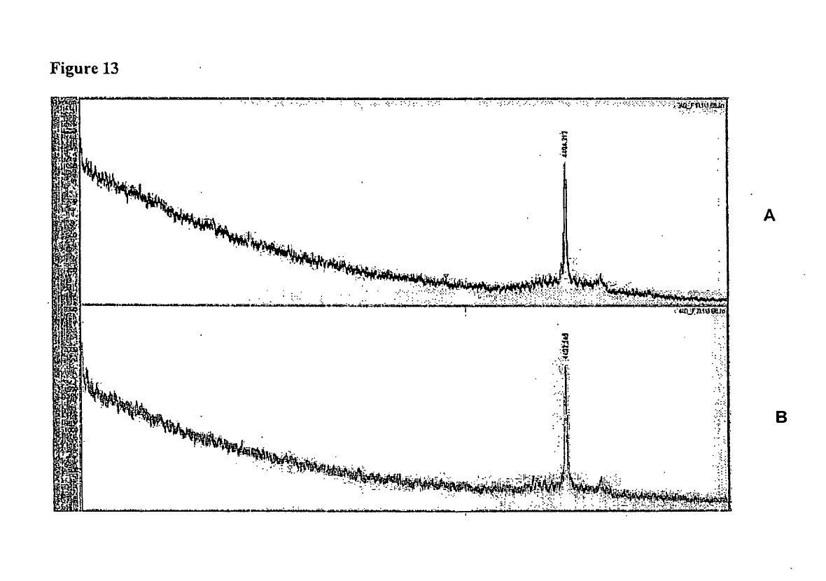

Spy1542 was therefore used to treat the WT strain challenged with 0.4 μM LL-37 in a process similar to the bacterial killing assay described above. After 30 minutes, the bacteria were pelleted and the supernatant was analysed. It was discovered by mass spectrometry that the LL-37 in the supernatant was unmodified by the action of Spy1542 (FIG. 13).

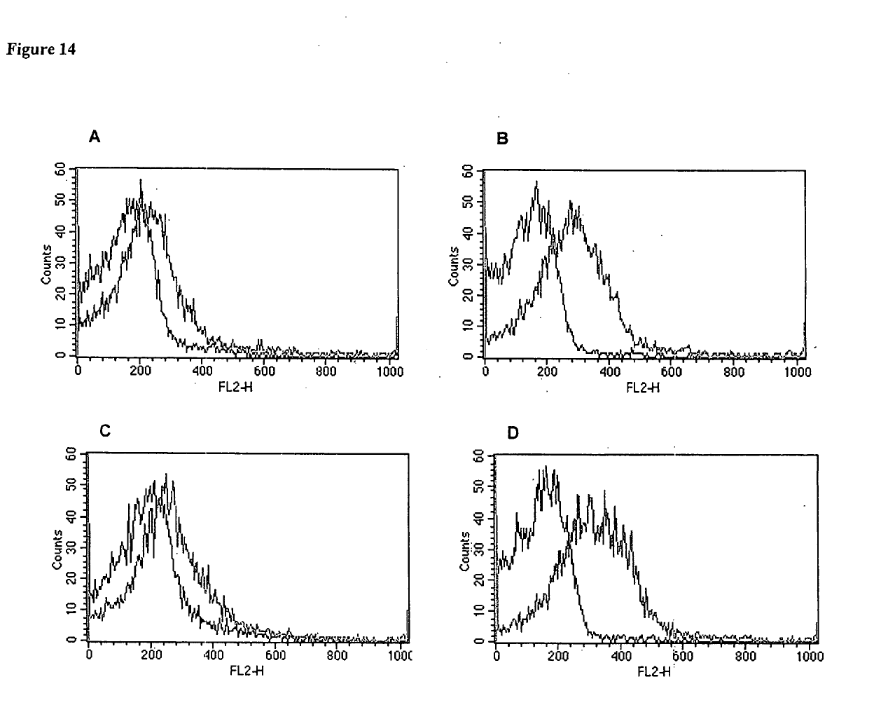

An experiment was carried out to see if LL-37 bound to the surface of the bacteria. 0.8 μM LL-37 was added to the bacteria in a method similar to that for the bacterial killing assay. The amount of LL-37 localised on the bacterial surface was then compared for WT and Δspy1542 mutant strains by FACS analysis. The results (FIG. 14) show that the WT strain had less LL-31 on the bacterial surface (as demonstrated by a smaller shift in the peaks of the two graphs showing 0 μM or 0.8 μM LL-37). This “protective” effect also increases with time. The WT strains therefore appear to be shielded against LL-37.

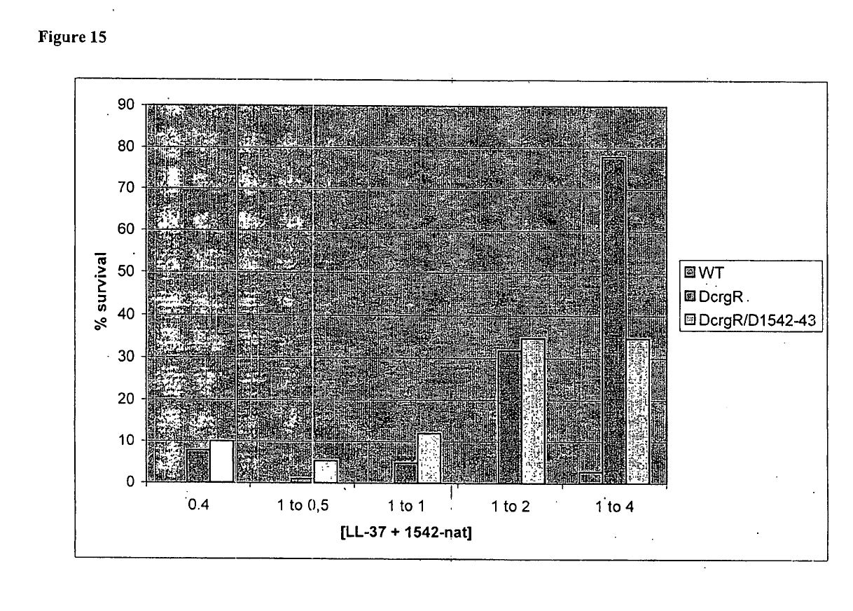

A further experiment was therefore carried out to confirm that the effect of Spy1542 was indirect. Spy1542 was added to the bacteria. The bacteria were then pelleted, washed and resuspended. 0.4 μM LL-37 was then added to the suspension. FIG. 15 shows that the protective effect of Spy1542 was maintained and that it was dose dependent.

It is postulated that the resistance to cathelicidins is due to electrostatic repulsion of the positively charged cathelicidins by positively charged peptidoglycan, produced by the activity of CrgE.

It will be understood that the invention has been described by way of example only and modifications may be made whilst remaining within the scope and spirit of the invention.

TABLE 1

| | | |
|---|---|---|
| Gene | fold | Annotation |

UPREGULATED

| | | |
|---|---|---|
| SPy2097 | 3.0 | PTS system, trehalose-specific IIBC component |
| dexS | 2.1 | putative dextran glucosidase (trehalose-6-phosphate hydrolase) |
| glgP | 3.3 | maltodextrin phosphorylase |
| malM | 1.9 | 4-alpha-glucanotransferase/amylomaltase/Disproportionating |
| | | enzyme/(malQ/dpeP) |
| malR | 1.1 | transcriptional regulator, LacI family, putative |
| SPy1294 | 3.9 | maltose ABC transporter, periplasmic maltose-binding protein, putative |
| malF | 2.3 | maltose ABC transporter, permease protein |
| malG | 2.7 | maltose ABC transporter, permease protein, putative |
| SPy1297 | 1.9 | regulator protein |
| SPy2111 | 1.0 | hypothetical protein |
| nrdD | 2.0 | putative anaerobic-ribonucleoside-triphosphate reductase |
| pfl | 6.8 | formate acetyltransferase |
| msmK | 1.3 | maltose/maltodextrin ABC transporter, ATP-binding protein |
| pulA | 2.7 | alkaline amylopullulanase |
| dexB | 2.4 | glucan 1,6-alpha-glucosidase |
| SPy0853 | 1.7 | glycerol-3 -phosphate regulon represser, putative |
| fruB | 1.8 | 1-phosphofructokinase, putative |
| SPy0855 | 2.3 | PTS system (phosphoenolpyruvate: sugar phosphotransferase system), |
| | | fructose-specific IIABC component |
| SPy1548 | 1.1 | Bacterial regulatory proteins, crp family domain protein |
| sagP (arcA) | 1.5 | arginine deiminase |
| SPy1546 | 1.2 | Acetyltransferase (GNAT) family family |
| arcB (SPy1544) | 1.8 | ornithine carbamoyltransferase |
| SPy1543 | 2.0 | conserved hypothetical transmembrane protein (putative arginine-ornithine |
| | | antiporter) |
| SPyl542 | 1.7 | putative Xaa-His dipeptidase |
| arcC | 1.3 | carbamate kinase |
| sagA | 2.7 | streptolysin S associated protein |
| sagB | 1.7 | Modifying enzyme |
| sagC | 2.7 | Unknown function |
| SPy0741 (sagD) | 1.5 | Uncharacterized ACR, COG 1944 superfamily |
| SPy0742 (sagE) | 3.2 | hypothetical protein (su Steiner K et al. 2001 si legge che sagE ha funzione di |
| | | cellular immunity) |
| SPy0743 (sagF) | 1.7 | hypothetical protein |
| SPy0744 (sagG) | 3.9 | ABC transporter, ATP-binding protein, putative |
| sagH | 4.5 | ABC transporter, putative |
| SPy0746 (sagI) | 1.6 | ABC transporter, putative |

DOWNREGULATED

| | | |
|---|---|---|
| opuAA | 0.5 | putative glycine betaine/proline ABC transporter (ATP-binding protein) |
| opuABC | 0.5 | putative glycine-betaine binding permease protein |
| scrA | 0.5 | PTS system (phosphoenolpyruyate: sugar phosphotransferase system), sucrose- |
| | | specific IIBC component |
| SPy1870 | 0.2 | Bacterial regulatory proteins, gntR family domain protein |

TABLE 2

| Construct name | Primer name | Primer sequence 5′-3′ |
|---|---|---|
| crgR construct | UDP F | SEQ ID NO: 7 |
| | CAT R | SEQ ID NO: 8 |
| | CAT F | SEQ ID NO: 9 |
| | GLY R | SEQ ID NO: 10 |
| sagAp construct | SAGAF1 | SEQ ID NO: 11 |
| | SAGAR1 | SEQ ED NO: 12 |
| | SAGAF2 | SEQ ID NO: 13 |
| | SAGAR2 | SEQ ID NO: 14 |
| Spy1542-3 construct | 1542-3F1 | SEQ ID NO: 15 |
| | 1542-3R1 | SEQ ID NO: 16 |
| | 1542-3F2 | SEQ ID NO: 17 |
| | 1542-3R2 | SEQ ID NO: 18 |

TABLE 3

| | Wild type | ΔcrgR | ΔcrgR/Δ1542-43 |
|---|---|---|---|
| | 0.350 | 0.080 | 0.020 |
| | 0.225 | 0.525 | 0.000 |
| | 0.050 | 0.225 | 0.020 |
| | 0.350 | 0.250 | 0.750 |
| | 0.080 | 0.105 | 0.200 |
| | 0.200 | 0.200 | 0.045 |
| | 0.225 | 0.400 | 0.000 |
| | 0.100 | 0.375 | 0.250 |
| GMT | 0.163 | 0.229 | 0.017 |
| Media | 0.198 | 0.270 | 0.161 |
| SD | 0.115 | 0.153 | 0.257 |

| | ΔcrgR/WT | ΔcrgR/Δ1542-43 |
|---|---|---|
| Ratio | 1.4 | 13.5 |

Lesion surface reported in cm2

### REFERENCES

### The Contents of which are Hereby Incorporated in Full

[1] Devine et al., 2002
[2] Gudmundsson et al, 1996
[3] Gallo et al, 1997
[4] Shai et al., 1999
[5] Henzler et al., 2003
[6] Dorschner R A et al. (2001) J. Invest. Dermatol. 117(1):91-7.
[7] Gallo et al, 1994
[8] Huang et al, 1997
[9] Risso et al, 1998
[10] Zaiou M & Gallo R L (2002) J. Mol. Med. 80(9):549-61
[11] Murakami et al., 2002
[12] Jeong et al., 2003
[13] Nizet et al. (2001) Nature 414(6862):454-7
[14] Toole B P (2000) J. Clin. Invest. 106(3):335-6
[15] Perez-Casal et al., 1993
[16] Nizet V & Gallo R L (2003) Scand J. Infect. Dis. 35:670-676
[17] Howell M D et al. (2004) J. Immunol. 172(3):1763-7
[18] Eisenthal & Danson (eds) Enzyme Assays (Practical Approach Series) ISBN:0199638209 (2002).
[19] Lin et al. (1997) Antimicrobial Agents and Chemotherapy 41:2127-2131.
[20] Gennaro (2000) Remington: The Science and Practice of Pharmacy. 20th edition, ISBN: 0683306472.
[21] WO01/30390.
[22] Almeida & Alpar (1996) J. Drug Targeting 3:455-467.
[23] Agarwal & Mishra (1999) Indian J Exp Biol 37:6-16.
[24] Bjune et al. (1991) Lancet 338(8775):1093-96
[25] WO01/52885.
[26] Fukasawa et al. (1999) Vaccine 17:2951-2958.
[27] Rosenqvist et al. (1998) Dev. Biol. Stand 92:323-333.
[28] Costantino et al. (1992) Vaccine 10:691-698.
[29] Costantino et al. (1999) Vaccine 17:1251-1263.
[30] WO03/007985.
[31] WO99/24578
[32] WO99/36544
[33] WO99/57280
[34] WO00/66791
[35] WO01/64922
[36] WO01/64920
[37] WO03/020756
[38] WO2004/032958
[39] WO2004/048404.
[40] Covacci & Rappuoli (2000) J. Exp. Med. 19:587-592.
[41] WO93/18150.
[42] Covacci et al. (1993) Proc. Natl. Acad. Sci. USA 90:5791-5795.
[43] Tummuru et al. (1994) Infect. Immun. 61:1799-1809.
[44] Marchetti et al. (1998) Vaccine 16:33-37.
[45] Telford et al. (1994) J. Exp. Med. 179:1653-1658.
[46] Evans et al. (1995) Gene 153:123-127.
[47] WO96/01272 & WO96/01273, especially SEQ ID NO:6.
[48] WO97/25429.
[49] WO98/04702.
[50] Watson (2000) Pediatr Infect Dis J 19:331-332.
[51] Rubin (2000) Pediatr Clin North Am 47:269-285.
[52] Jedrzejas (2001) Microbiol Mol Biol Rev 65:187-207.
[53] Bell (2000) Pediatr Infect Dis J 19:1187-1188.
[54] Iwarson (1995) APMIS 103:321-326.
[55] Gerlich et al (1990) Vaccine 8 Suppl:S63-68 & 79-80.
[56] Hsu et al. (1999) Clin Liver Dis 3:901-915.
[57] Stratov et al. (2004) Curr Drug Tgts 5(1):71-88.
[58] Vaccines (1988) eds. Plotkin & Mortimer. ISBN 0-7216-1946-0.
[59] Gustafsson et al. (1996) N. Engl. J. Med. 334:349-355.
[60] Rappuoli et al. (1991) TIBTECH 9:232-238.
[61] Sutter et al. (2000) Pediatr Clin North Am 47:287-308.
[62] Zimmerman & Spann (1999) Am Fam Physician 59:113-118, 125-126.
[63] WO99/24578.
[64] WO99/36544.
[65] WO99/57280.
[66] WO02/079243.
[67] WO02/02606.
[68] Kalman et al. (1999) Nature Genetics 21:385-389.
[69] Read et al. (2000) Nucleic Acids Res 28:1397-406.
[70] Shirai et al. (2000) J. Infect. Dis. 181 (Suppl 3):S524-S527.
[71] WO99/27105.
[72] WO00/27994.
[73] WO00/37494.
[74] WO99/28475.
[75] Ross et al. (2001) Vaccine 19:4135-4142.
[76] Dreesen (1997) Vaccine 15 Suppl:S2-6.
[77] MMWR Morb Mortal Wkly Rep 1998 Jan. 16; 47(1):12, 19.
[78] Anderson (2000) Vaccine 19 Suppl 1:S59-65.
[79] Kahn (2000) Curr Opin Pediatr 12:257-262.
[80] Crowe (1995) Vaccine 13:415-421.
[81] McMichael (2000) Vaccine 19 Suppl 1:S101-107.
[82] WO02/34771.
[83] Dale (1999) Infect Dis Clin North Am 13:227-43, viii.
[84] Ferretti et al. (2001) PNAS USA 98:4658-4663.
[85] Kuroda et al. (2001) Lancet 357(9264):1225-1240; see also pages 1218-1219.
[86] Modlin et al. (2001) J Toxicol Clin Toxicol 39:85-100.
[87] Demicheli et al. (1998) Vaccine 16:880-884.
[88] Stepanov et al. (1996) J Biotechnol 44:155-160.
[89] Jiang et al. (2005) Emerg. Infect. Dis. 11(7):1016-20.
[90] Estes et al. (2000) J Infect Dis. 181 Suppl 2:S367-73.
[91] Ingram (2001) Trends Neurosci 24:305-307.
[92] Rosenberg (2001) Nature 411:380-384.
[93] Moingeon (2001) Vaccine 19:1305-1326.

1. A Streptococcus bacterium in which expression of CrgE and/or CrgR has been knocked out.

2. A bacterium according to claim 1, wherein said bacterium is Streptococcus pyogenes.

3. A polypeptide comprising the amino acid sequence of SEQ ID NO: 2 or SEQ ID NO: 4.

4. A polypeptide comprising an amino acid sequence: (a) having sequence identity to an amino acid sequence selected from SEQ ID NO: 2 or SEQ ID NO: 4 and/or (b) comprising a fragment of an amino acid sequence selected from SEQ ID NO: 2 or SEQ ID NO: 4.

5. A fusion protein comprising a CrgE or CrgR polypeptide.

6. A mutant CrgE polypeptide that cannot bind its substrate.

7. (canceled)

8. A method of preventing and/or treating graft rejection comprising administering CrgE polypeptide to a subject at risk for graft rejection.

9. (canceled)

10. A method of enhancing the immune response to a viral vaccine comprising administering CrgE as an adjuvant.

11. A method of enhancing vaccination comprising administering a composition comprising a vaccine and CrgE to a subject.

12. A process for determining whether a test compound down-regulates the expression of CrgE polypeptide, comprising the steps of (a) contacting the test compound with a S. pyogenes bacterium to form a mixture; (b) incubating the mixture to allow the compound and the bacterium to interact; and (c) determining whether expression of the polypeptide is down-regulated.

13. A process for determining whether a test compound binds to a CrgE or CrgR polypeptide, comprising the steps of (a) contacting the test compound with the polypeptide to form a mixture; (b) incubating the mixture to allow the compound and the polypeptide to interact; and (c) determining whether the compound and polypeptide interact.

14. A process for determining whether a test compound inhibits the enzymatic activity of a CrgE or CrgR polypeptide, comprising the steps of: (a) contacting the test compound with the polypeptide and a substrate for the enzymatic reaction catalysed by the polypeptide; (b) incubating the mixture to allow the compound, polypeptide and substrate to interact; and (c) determining whether modification of the substrate by the enzymatic activity of the polypeptide is inhibited by the test compound.

15. A compound identified by the process of any one of claims 12-14.

16. A method for detecting a bacterium expressing a CrgE or CrgR polypeptide, comprising the steps of: (a) contacting a S. pyogenes bacterium with an antibody specific for CrgR or CrgE; (b) incubating the mixture to allow the antibody and bacterium to interact; and (c) detecting binding of the antibody to the bacterium.

17. A method for detecting a bacterium expressing a CrgE or CrgR polypeptide, comprising the steps of: (a) contacting a S. pyogenes bacterium with a first antibody specific for CrgR or CrgE; (b) incubating the mixture to allow the first antibody and bacterium to interact; (c) contacting the mixture with a second labelled antibody specific for the first antibody; and (d) detecting binding of the second labelled antibody to the first antibody.

18. A pharmaceutical composition comprising CrgE polypeptide or a homologue thereof.

19. A composition according to claim 18 further comprising a pharmaceutically acceptable carrier.

20. (canceled)

21. (canceled)

22. A method of enhancing an immune response in a mammal comprising administering an effective amount of a composition according to claim 18 or claim 19.


SEQUENCE LISTING (20 sequences)

SEQ ID NO: 1 - DNA, 744 bp, Streptococcus pyogenes
atgtctactaacgacttaaccaaaaaactcaaaaaacttaaacatgtgcaagtatataatactatttttcaacttattcaagatggtacatatagtcctggcatgcaactgccatcagaacctgaacttgctagacagctcaatgtcagtcggatgacactacgtaaatccctagccctcctccaagaggatcatctcataaaaaatatcagaggaaagggaaattttatactcaagactcctgaaactaaatatcaccaaggtttcgaatatcttcaacaccccatatatgcaagcctatcatctgacatcacaaaggttgagttggaatatcggattgaagtgcccactgttgccattacagcatccctaaaacaagaaactcctgttgtgattattgttgatcgctggtatcatagccaaaataaagctattgcttatagtttatcttttatccctattgaggttatttctaaatatgctataaatctcaatcaagaagagccccttcttactttcttagaagagaaaatctatgaatctggtaaagcttctcattcctgcaaccaaatcggctataccaagactggcaattacacagcaactaagtatactctatcagaaaatagtgcttttattttaatccaagaaactctctacaatggtaaagacatcttggtctcaaccaaacactacgttcctgctgatttatttgacttaaaagttcaatctcaaagttgctaa

SEQ ID NO: 2 - protein, 247 aa, Streptococcus pyogenes
MSTNDLTKKLKKLKHVQVYNTIFQLIQDGTYSPGMQLPSEPELARQLNVSRMTLRKSLALLQEDHLIKNIRGKGNFILKTPETKYHQGFEYLQHPIYASLSSDITKVELEYRIEVPTVAITASLKQETPVVIIVDRWYHSQNKAIAYSLSFIPIEVISKYAINLNQEEPLLTFLEEKIYESGKASHSCNQIGYTKTGNYTATKYTLSENSAFILIQETLYNGKDILVSTKHYVPADLFDLKVQSQSC

SEQ ID NO: 3 - DNA, 1332 bp, Streptococcus pyogenes
atggaatcctatatcacacctaaacatcaagaggcctgtgtagctgccattaaacagatcgtttcttacccttctgtttgtcatgaaggagaaaatggcacaccttttggacaggccattcaagatattctcgaagcaaccttagacttatgtcaaggtcttggttttcatacttatattgatcctgaaggttactatggctatgctgagttaggtgaccaaaaagaggtcttggctattctttgccatttggacgttgttccagaaggagatcgtcagttatggaagacagatccttttgactgtgttgaggcagacggctgtttatttggtcgaggcactcaagacgataaagggcctactatgatggcgctatttgccaccaaagccttgatagatgcaggcgttacctttaataaacgcattcgttttattttcggaacagatgaagaaactttgtggcggtgtatgaatcgctacaatgatgtagaagaacaggctacctttgggtttgctcctgattcaagtttccctttaacctatgccgaaaaaggcctacttcaggcaaaattagtggggaaaggttctgataaactgtcacttgaaatcggacaagcctacaatgttgttcctgcaagagcctcttatcaaggtgataagcttgaggctttacaaaaagaactagataagcttggctttgagtacgtcgtcaaagagggagaattaacggtttatggtctcgcccaacatgctaaagatgctcccgatggcatcaacgctatgattaggcttgccaaggctttagtggtccttgaacctaacaaaacccttgattttcttgctaatgtcattgatgaagatggcagagcacttaatatttttggcagaatcgaagatgaaccttcaggcaaattaagctttaatgccgcagggcttaccttaaccaaagacaaggcagaaattcgcctagatattcggattccagttttggcagataaggaaaaattagtccaacaattaagccaaaaagcacaagaatacggcctaacctatgaagaatttgattatttggccccattatatgtgccattagacagtgaattggtaacaaccttgttatctgtctatcggaaaaaaacaggtgatcaaagccctgcccaatcatcaggaggggcaacctttgctagaacaatgaagaattgtgttgcctttggtgcccttttccctgatgctgttcaaacggaacaccaagaaaatgaacatattgttttagcggatgcttaccgcgctatggatatttatgcggaagctatttaccgactaacgcgataa

SEQ ID NO: 4 - protein, 443 aa, Streptococcus pyogenes
MESYITPKHQEACVAAIKQIVSYPSVCHEGENGTPFGQAIQDILEATLDLCQGLGFHTYIDPEGYYGYAELGDQKEVLAILCHLDVVPEGDRQLWKTDPFDCVEADGCLFGRGTQDDKGPTMMALFATKALIDAGVTFNKRIRFIFGTDEETLWRCMNRYNDVEEQATFGFAPDSSFPLTYAEKGLLQAKLVGKGSDKLSLEIGQAYNVVPARASYQGDKLEALQKELDKLGFEYVVKEGELTVYGLAQHAKDAPDGINAMIRLAKALVVLEPNKTLDFLANVIDEDGRALNIFGRIEDEPSGKLSFNAAGLTLTKDKAEIRLDIRIPVLADKEKLVQQLSQKAQEYGLTYEEFDYLAPLYVPLDSELVTTLLSVYRKKTGDQSPAQSSGGATFARTMKNCVAFGALFPDAVQTEHQENEHIVLADAYRAMDIYAEAIYRLTR

SEQ ID NO: 5 - protein, 173 aa, Mus musculus
MQFQRDVPSLWLWRSLSLLLLLGLGFSQTPSYRDAVLRAVDDFNQQSLDTNLYRLLDLDPEPQGDEDPDTPKSVRFRVKETVCGKAERQLPEQCAFKEQGVVKQCMGAVTLNPAADSFDISCNEPGAQPFRFKKISRLAGLLRKGGEKIGEKLKKIGQKIKNFFQKLVPQPEQ

SEQ ID NO: 6 - protein, 170 aa, Homo sapiens
MKTQRDGHSLGRWSLVLLLLGLVMPLAIIAQVLSYKEAVLRAIDGINQRSSDANLYRLLDLDPRPTMDGDPDTPKPVSFTVKETVCPRTTQQSPEDCDFKKDGLVKRCMGTVNLNQARGSFDISCDKDNKRFALLGDFFRKSKEKIGKEFKRIVQRIKDFLRNLVPRTES

SEQ ID NO: 7 - DNA, 21 bp, Artificial Sequence
  Primer UDP F
caacagcaacttgaatagctg

SEQ ID NO: 8 - DNA, 41 bp, Artificial Sequence
  Primer CAT R
aatcagcaggaacgtagtgtaggactatatgtaccatcttg

SEQ ID NO: 9 - DNA, 41 bp, Artificial Sequence
  Primer CAT F
caagatggtacatatagtcctacactacgttcctgctgatt

SEQ ID NO: 10 - DNA, 21 bp, Artificial Sequence
  Primer GLY R
tgataacagaagaccacttag

SEQ ID NO: 11 - DNA, 20 bp, Artificial Sequence
  Primer SAGAF1
ggtactgtagctatccgttc

SEQ ID NO: 12 - DNA, 43 bp, Artificial Sequence
  Primer SAGAR1
caacaagtagtacagcagcaacgaatcaatcaaggttgtagag

SEQ ID NO: 13 - DNA, 43 bp, Artificial Sequence
  Primer SAGAF2
ctctacaaccttgattgattcgttgctgctgtactacttgttg

SEQ ID NO: 14 - DNA, 22 bp, Artificial Sequence
  Primer SAGAR2
cagcattaatagcaccgtattc

SEQ ID NO: 15 - DNA, 22 bp, Artificial Sequence
  Primer 1542-3F1
cgatagacagataacaaggttg

SEQ ID NO: 16 - DNA, 43 bp, Artificial Sequence
  Primer 1542-3R1
tggtacttggtatttcccagaacttctgtttgtcatgaaggag

SEQ ID NO: 17 - DNA, 43 bp, Artificial Sequence
  Primer 1542-3F2
ctccttcatgacaaacagaagttctgggaaataccaagtacca

SEQ ID NO: 18 - DNA, 20 bp, Artificial Sequence
  Primer 1542-3R2
ctacatgacaagtcgttctg

SEQ ID NO: 19 - DNA, 273 bp, Streptococcus pyogenes
attaaaggtcagaacactttattttaaaagtttttctgacttattttaatttataagtataaatttttacactaagatgtaaaaaacatacaagtaaacgaatacaaatctataaaaataaaaacaacgctcaaaaaatgtaagcgattactcaaaatagctgtgagaaataccctatttttatcttttttaaggagtacactaggaatataagcaagagttaagaatcttgacggtgaattcacaaaaaccaattagaaagaggtaattctt

SEQ ID NO: 20 - DNA, 170 bp, Streptococcus pyogenes
taaggactggtactccttaactcatttcacatatcaatgtgacccctttaatttaacaatttaactacttaaaaacgtactttaactacatgacaagtcgttctggttttcgcgtccttgaccggaactagaacggcttatttttatgaaaaggtaagaggtaatcatta